(19) United States
(12) Patent Application Publication
(10) Pub. No.: US 2008/0249298 A1
(43) Pub. Date: Oct. 9, 2008

(54) USE OF HEPARINOID DERIVATIVES FOR THE TREATMENT AND DIAGNOSIS OF DISORDERS WHICH CAN BE TREATED WITH HEPARINOIDS

(75) Inventors: Wolfgang ULMER, Frankfurt am Main (DE); Hans-Paul JURETSCHKE, Frankfurt am Main (DE); Christopher KERN, Frankfurt am Main (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt am Main (DE)

(21) Appl. No.: 12/142,233

(22) Filed: Jun. 19, 2008

Related U.S. Application Data
(63) Continuation of application No. 10/223,145, filed on Aug. 19, 2002, now abandoned.

(30) Foreign Application Priority Data
Aug. 22, 2001 (DE) 10141106.5

(51) Int. Cl. C07H 23/00

(52) U.S. Cl. 536/121

(57) ABSTRACT

Heparinoid derivatives comprising a chelating agent which is covalently bonded to the heparinoid, and a paramagnetic metal cation from the series of transition metals Sc, Ti, V, Cr, Mn, Fe, Co, Ni, Cu, Mo, Ru or of the lanthanides, are suitable for producing medicaments both for therapy and for diagnostic purposes, for localizing the dose employed, and for monitoring the result of treatment of disorders such as thrombosis and osteoarthrosis.

The present invention relates to heparinoid derivatives, processes for their preparation and their use both in therapy and for diagnostic purposes, for localization of the dose employed, and for monitoring the result of treatment of disorders such as thrombosis and osteoarthrosis.

Heparin is a highly sulfonated glycosaminoglycan which can be isolated from animal organs, is synthesized in mast cells and consists of D-glucosamine and D-glucuronic acid, having a molecular weight of about 17,000 daltons.

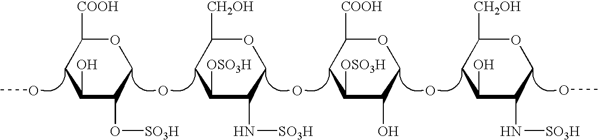

This involves the α-1,4-glycosidic linkage of D-glucosamine and D-glucuronic acid to give the disaccharide, and linking the heparin subunits likewise with α-1,4-glycosidic linkages with one another to form heparin. The position of the sulfo groups may vary; a tetrasaccharide unit contains 4 to 5 sulfate residues. Heparan sulfate (heparitin sulfate) contains fewer O- and N-bonded sulfo groups but also contains N-acetyl groups. Heparin can be regarded as an anionic polyelectrolyte. Heparin occurs, bound to proteins, especially in the liver (Greek: hepar) and, as anticoagulant, prevents coagulation of the blood circulating in the body. Heparan sulfate is found as a constituent of proteoglycans (perlecan) on cell surfaces and in the extracellular matrix of many tissues. Heparin intensifies the inhibitory effect of antithrombin III on thrombin, which blocks the catalysis of the conversion of fibrinogen into fibrin by thrombin, and on various other coagulation factors; for example, the conversion of prothrombin into thrombin is also prevented and breakdown of lipoprotein by lipoprotein lipase is activated.

Heparinoid is a collective term for all substances which have heparin-like effects. These include pentosan polysulfate, xylan sulfates, dextran sulfates or chitin sulfates, di-, tri-, or oligomers and polymers of iduronic/uronic acids and/or glucosamine, oligo- or polysaccharides composed of pentose and/or hexose units and/or mannitol in random or regular arrangement, heparan sulfates, heparitin sulfates, keratan sulfates or dermatan sulfates, hyaluronic acid, chondroitin sulfate A, B or C, unfractionated heparin and fractionated heparin, or synthetic polysaccharides comparable thereto, and the salts thereof, and linked and crosslinked chains (di-, tri- or oligomers) of the abovementioned compounds, and heparinoids with peptides, proteins, lipids or nucleic acids bound thereto. Fractionated heparins include enoxaparin, nadroparin (Fraxiparin), dalteparin (Fragmin®), bemiparin, tinzaparin, ardeparin, low molecular weight heparin (LMWH), and ultra low molecular weight heparin (ULMWH).

Enoxaparin is an active ingredient which belongs to the class of low molecular weight heparins (LMWH) as disclosed in patents of Aventis Pharma, such as U.S. Pat. No. 5,389,618. The use of enoxaparin for antithrombotic therapy is established in the art. Enoxaparin-Na is the sodium salt of low molecular weight heparin which is obtained by alkaline depolymerization of the benzyl ester derivative of heparin from porcine intestinal mucosa. The major amount of the components of a 4-enopyranose uronate structure are at the nonreducing end of the chain thereof. The average molecular mass is about 4,500 daltons. The percentage content of molecules of less than 2,000 daltons is between 12% and 20%. The mass percentage content of chains with a size between 2,000 and 8,000 daltons is between 68% and 88% based on the European Pharmacopoeia calibration reference standard for low molecular weight heparins. The degree of sulfation averages 2 residues per disaccharide unit. The enoxaparin polysaccharide chain is, as in heparin, composed of alternating units of sulfated glucosamines and uronic acids, which are linked by glycosidic bonds. The structure differs from heparin for example in that the depolymerization process results in a double bond at the nonreducing end of the chain. Enoxaparin can be distinguished from heparin by UV spectroscopy and by the ¹³C nuclear magnetic resonance spectrum, which show the double bond in the terminal ring, and by high performance size exclusion chromatography.

In the pathological state of osteoarthrosis, degradation of the aggrecan, the main proteoglycan of articular cartilage, represents a very early and crucial event. The pathological loss of the cartilage aggrecan results from proteolytic cleavages in its interglobular domain. Amino acid sequence analyses of proteoglycan metabolites isolated from the synovial fluid of patients suffering from joint damage, osteoarthrosis or an inflammatory joint disorder have shown that a proteolytic cleavage takes place preferentially between the amino acids Glu³⁷³ and Ala³⁷⁴ in the interglobular domain of human aggrecan (Lohmander et al., Arthritis Rheum. 36, (1993), 1214-1222). The proteolytic activity responsible for this cleavage is referred to as “aggrecanase” and may be assigned to the superfamily of metalloproteinases (MP).

Zinc is essential in the catalytically active site of metalloproteinases. MPs cleave collagen, laminin, proteoglycans, elastin or gelatin under physiological conditions and therefore play an important role in bone and connective tissue. A large number of different MP inhibitors are known (J. S. Skotnicki et al., Ann. N.Y. Acad. Sci. 878, 61-72 [1999]; EP 0 606 046; WO94/28889). Some of these inhibitors are not well characterized in relation to their specificity; others are more or less selectively directed in particular against matrix metalloproteinases (MMPs).

Aggrecanase differs from matrix metalloproteinases (MMPs) by different specificity, which is directed against particular cleavage sites which occur in aggrecan and are favored by MMPs. The cleavage results in characteristic fragments which can be detected by using suitable antibodies.

It was found in previous works that enoxaparin inhibits dose-dependently the aggrecanase activity in synovial fluid and, therefore, can be employed for the therapy of osteoarthrosis by intra-articular administration (DE 100 63 006.5).

An inherent disadvantage of heparinoids (polysaccharides) is, however, that they do not contain in their chemical structure a chromophore which can be used for analysis. The consequence of this is that analytical monitoring of therapeutically active concentrations by conventional analytical methods such as high-pressure liquid chromatography (HPLC) encounters considerable problems. It is usually possible to monitor antithrombotic therapy with heparinoids with adequate sensitivity only through the biological effect thereof, the inhibition, catalyzed by heparinoids, of factor Xa by antithrombin III. This disadvantage of therapy with heparinoids is particularly unsatisfactory and inconvenient if interest is centered on effects of the heparinoids other than their anticoagulant effect, or if in the case of locally restricted therapy the exact concentration of the heparinoid at the site of action, its residence time and its distribution behavior must be known, as is necessary, for example, with intra-articular injection for the therapy of osteoarthrosis or for use for stroke, angina pectoris, embolisms or in tumor therapy.

On the other hand, a fundamental problem with all known therapies of osteoarthrosis is the difficulty of diagnosing the result of treatment in the affected cartilage and bone tissues.

It has now been found that the heparinoid derivatives of the present invention are able to eliminate said disadvantages.

The heparinoid derivatives of the present invention are distinguished by
acting as strong inhibitors on the activity of aggrecanase, hADAMTS1 and gelatinase A (MMP −2) and, therefore, being suitable for the therapy of osteoarthrosis,
showing as anticoagulant medicinal substances a comparable antithrombotic effect to heparin or enoxaparin,
but at the same time, in contrast to heparinoids, being directly observable at the site of action by magnetic resonance imaging methods (MRI), so that the local concentration after administration can be monitored and the distribution behavior of the medicament in the patient can be followed. The heparinoid derivatives of the invention are for this reason also suitable, in particular, for use in cases of stroke, angina pectoris, embolism and in tumor therapy. They are, however, also extremely suitable in cases of osteoarthrosis for gaining diagnostic information about the condition of the diseased connective tissue from the cartilage penetration behavior during the therapy.

The heparinoid derivatives of the present invention comprise a chelating agent which is covalently bonded to a heparinoid, and a paramagnetic metal cation from the series of transition metals Sc, Ti, V, Cr, Mn, Fe, Co, Ni, Cu, Mo, Ru or of lanthanides La, Ce, Pr, Nd, Pm, Sm, Eu, Gd, Tb, Dy, Ho, Er, Tm, Yb. Preferred heparinoids are, for example, enoxaparin or heparin.

Preference is further given to a heparinoid derivative which comprises as chelating agent diethylenetriamine-N,N,N′,N″,N″-pentaacetic acid dianhydride (DTPA), 1,2-bis(2-aminoethoxyethane)-N,N,N′,N′-tetraacetic acid (EGTA), ethylenediamine-N,N,N′,N′-tetraacetic acid (EDTA), 1,4,7,10-tetraazacyclododecane-1,4,7,10-tetraacetic acid (DOTA), 1,4,7,10-tetraazacyclododecane-1,4,7-triacetic acid (DO3A), nitrilotriacetic acid (NTA), triethylenetetraminehexaacetic acid (TTHA), 4-carboxy-5,8,11-tris(carboxymethyl)-1-phenyl-2-oxa-5,8,11-triazamidecan-13-oic acid (BOPTA) or N,N′-bis(pyridoxal 5-phosphate) ethylenediamine-N,N′-diacetic acid (DPDP). Anhydrides of the chelating agents are preferably employed for the chemical modification, particularly preferably diethylenetriamine-N,N,N′,N″,N′″-pentaacetic acid dianhydride (DTPA anhydride).

A particularly preferred paramagnetic metal cation is Gd³⁺, employed in the form of its salts gadolinium(III) chloride hexahydrate or gadolinium(III) acetate hydrate.

The invention further includes heparinoid derivatives in which the content of transition element or lanthanide may range from 1 mol per mol of heparinoid up to the maximum possible derivatization of the heparinoid, preferably from 1 mol to 20 mol per mol of heparinoid.

The invention also relates to a process for preparing the heparinoid derivatives of the invention, which comprises reacting the heparinoid with an activated chelating agent to give a heparinoid chelate, and then adding the transition element or lanthanide.

A procedure for preparing the heparinoid derivatives of the invention is, for example, first dissolving the heparinoid in a buffer. Suitable buffers have a pH of from 6.0 to 10.0, preferably pH 8.8. The concentration of the buffer is from 0.01 to 0.5 molar, preferably 0.1 molar. Examples of suitable buffers are carbonate buffers, borate buffers or biological buffers based on sulfonic acids, preferably HEPES (N-(2-hydroxyethyl)piperazine-N′-(2-ethanesulfonic acid)). An activated chelating agent is then added, for example DTPA anhydride. The activated chelating agent can be added in solid form or as solution. The reaction takes place at a temperature of from 8° C. to 37° C., preferably 24° C. The pH is preferably kept constant during the reaction.

The ratio of activated chelating agent to heparinoid is from 1:1 to 50:1, preferably from 1.5:1 to 15:1, based on the molecular weight.

After this, preferably without further purification of the heparinoid chelate, the transition element or lanthanide is added. The complexation takes place at a temperature of from 0° C. to 37° C., preferably 4° C. The pH is changed to a weakly acidic value and is then preferably left constant at this. The pH is 6.8 to 5, preferably pH 6.5.

The ratio of heparinoid chelate to the transition element or lanthanide is from 1:1 to 1:50, preferably from 1:1.5 to 1:15, based on the molecular weight.

The resulting heparinoid derivative of the invention can be further purified depending on the intended use. For example, the salt can be removed by dialysis or gel filtration. The resulting product can then be freeze-dried.

Enoxaparin and physiologically tolerated salts of enoxaparin are known and can be prepared as described, for example, in U.S. Pat. No. 5,389,618. They are mixtures of sulfated polysaccharides with the basic structure of the polysaccharides forming heparin, which are characterized by having an average molecular weight of about 4,500 daltons, which is lower than that of heparin, by comprising between 9% and 20% chains with a molecular weight of less than 2,000 daltons and only between 5% and 20% chains with a molecular weight of more than 8,000 daltons, and by the ratio of weight average molecular weight to number average molecular weight in them being between 1.3 and 1.6.

The invention also relates to medicaments having an effective content of at least one heparinoid derivative and/or a physiologically tolerated salt of the heparinoid derivative together with a pharmaceutically suitable and physiologically tolerated carrier, additive and/or other active ingredients and excipients.

Physiologically tolerated salts are prepared from heparinoid derivatives capable of salt formation in the manner known per se. The carboxylic acids form stable alkali metal, alkaline earth metal or, where appropriate, substituted ammonium salts with basic reagents such as hydroxides, carbonates, bicarbonates, alcoholates, and ammonia or organic bases, for example, trimethylamine or triethylamine, ethanolamine or triethanolamine or else basic amino acids, for example lysine, ornithine or arginine. Where the heparinoid derivatives have basic groups, it is also possible to prepare stable acid addition salts with strong acids. Suitable for this purpose are both inorganic and organic acids, such as, for example, hydrochloric, hydrobromic, sulfuric, phosphoric, methanesulfonic, benzenesulfonic, p-toluenesulfonic, 4-bromobenzenesulfonic, cyclohexylsulfamic, trifluoromethylsulfonic, acetic, oxalic, tartaric, succinic or trifluoroacetic acid.

The invention also relates to a process for producing a medicament, which comprises making a suitable dosage form from the heparinoid derivative of the invention with a pharmaceutically suitable and physiologically tolerated carrier and, where appropriate, other suitable active ingredients, additives or excipients.

Excipients which are frequently used and which may be mentioned are lactose, mannitol and other sugars, magnesium carbonate, lactalbumin, gelatin, starch, cellulose and its derivatives, animal and vegetable oils such as fish liver oil, sunflower, peanut or sesame oil, polyethylene glycol, and solvents such as sterile water, dimethyl sulfoxide (DMSO) and monohydric or polyhydric alcohols such as, for example, glycerol.

Because of the pharmacological properties, the heparinoid derivatives of the invention are suitable for the prophylaxis and therapy of all disorders in the course of which an increased catabolic activity of proteinases such as metalloproteinases plays a crucial part.

This is the case, for example, in degenerative joint disorders such as osteoarthroses, spondyloses, chondrolysis after joint trauma or prolonged immobilization of joints following meniscus or patella injuries or torn ligaments; also in disorders of connective tissue such as collagenoses, periodontal disorders, disturbances of wound healing and chronic disorders of the locomotor system such as inflammatory, immunology- or metabolism-related acute and chronic arthritis, arthropathies, myalgias and disturbances of bone metabolism.

The heparinoid derivatives of the invention can likewise be employed advantageously as antithrombotic agents. They can be used in particular for preventing venous thromboses in risk situations. This also applies to situations where the risk is chronic. The heparinoid derivatives of the invention make it possible in particular to reduce, with fixed doses, the risks of thrombotic events in orthopedic surgery.

An advantageous therapeutic use of heparinoid derivatives of the invention is based on the prevention of arterial thrombotic events, in particular in the event of a myocardial infarction, associated with unstable angina pectoris or recurrent angina. A further interesting use of the heparinoid derivative of the invention is based on the possibility of using them to prevent venous thromboses in surgical patients postoperatively. This use is exceptionally advantageous because it permits the risks of a hemorrhage to be avoided during the operation. It is equally advantageous to use the heparinoid derivatives of the invention after angiography and in therapy of stenosis and restenosis.

Further possible applications of the heparinoid derivatives of the invention relate to advantageous effects in tumor and metastasis therapy in oncology (antiproliferative effects), in the therapy of inflammatory disorders (antiinflammatory effect), for disorders of the central nervous system (CNS), and for transplants. The administration is likewise possible for ischemias associated with myocardial and cerebral infarctions (reduction of infarct size), for asthma (effect on tryptase) or angiogenesis (promoting effect of FGF-mediated cell proliferation).

The heparinoid derivatives of the invention are generally administered parenterally. It can take place by subcutaneous, intra-articular, intraperitoneal or intravenous injection. Rectal, oral, inhalation or transdermal administration is likewise possible. Intra-articular injection is preferred for osteoarthrosis.

The pharmaceutical products are preferably produced and administered in dosage units, each unit comprising as active ingredient a particular dose of the heparinoid derivative of the invention. This dose can be from about 0.5 μg to about 200 mg for injection solutions in ampoule form, preferably from about 10 mg to 80 mg for systemic administration, and preferably 1 μg to 10 mg for local administration.

The invention also relates to the use of the heparinoid derivatives of the invention for monitoring and diagnosis of the progress of disorders whose course involves an increased activity of metalloproteinases.

The invention also relates to the use of the heparinoid derivatives of the invention for producing a diagnostic test system. The invention further relates to the use of such a diagnostic test system for monitoring the result of treatment and functional characterization of disorders.

### EXAMPLE 1

### Preparation of Enoxaparin

### 1. Esterification

15 ml of benzyl chloride are added to a solution of 15 g of benzethonium heparinate in 75 ml of methylene chloride. The solution was heated to a temperature of 35° C., which was maintained for 25 hours. Then 90 ml of a 10% strength sodium acetate solution in methanol were added, followed by filtration, washing with methanol and drying. This resulted in 6.5 g of heparin benzyl ester in the form of the sodium salt, and the degree of esterification thereof, determined as indicated above, was 13.3%.

### 2. Depolymerization

10 g of the heparin benzyl ester obtained above, in the form of the sodium salt, were dissolved in 250 ml of water. This solution was heated to 62° C. and 0.9 g of sodium hydroxide solution was added. The temperature was kept at 62° C. for 1 hour and 30 minutes. The reaction mixture was then cooled to 20° C. and neutralized by adding dilute hydrochloric acid. The concentration of the reaction medium was then adjusted to 10% in sodium chloride. The product was finally precipitated in 750 ml of methanol, filtered and dried. This resulted in a heparin with the following structural features:

average molecular weight about 4,500 daltons
molecular distribution:
20% chains with a molecular weight of less than 2,000 daltons
5.5% chains with a molecular weight of more than 8,000 daltons
Dispersion: d=1.39

### EXAMPLE 2

### Preparation of an Enoxaparin Derivative (En-15)

100 mg of enoxaparin (solid, prepared as described in Example 1) were dissolved in 5 ml of a 0.1 molar HEPES buffer pH 8.8. While stirring at 24° C., a prepared suspension prepared from 119 mg of diethylenetriamine-N,N,N′,N″,N″-pentaacetic acid dianhydride (DTPA anhydride) and 0.34 ml of dimethyl sulfoxide, corresponding to a 15-fold molar excess of reagent over the amount of enoxaparin introduced, was added dropwise.

During the addition, the pH was monitored and kept at pH 8.8 by metering in 1 molar sodium hydroxide solution. The reaction mixture was vigorously stirred at room temperature for 30 minutes, keeping the pH constant during this, if necessary, by further addition of 1 molar sodium hydroxide solution. 123.8 mg of solid gadolinium(III) chloride hexahydrate, likewise corresponding to a 15-fold molar excess over the enoxaparin present, were then added to the mixture. The pH was adjusted to 6.5 by titration with 1 N hydrochloric acid. The reaction mixture was stirred further at 4° C. for 24 hours.

The modified enoxaparin fraction was desalted and separated from unreacted reagent by gel filtration on Sephadex G-25®. It is also possible to employ for this purpose commercially available Pharmacia PD-10® prepacked columns in accordance with the manufacturer's description. The modified enoxaparin was freeze-dried.

91 mg of a modified enoxaparin were obtained. The factor Xa inhibition test showed an inhibitory strength comparable with that of the original enoxaparin. Analysis of the gadolinium incorporation by inductively coupled plasma atomic emission spectrometry revealed an average content of 2 mol of gadolinium per mol of enoxaparin.

### EXAMPLE 3

### Preparation of an Enoxaparin Derivative (EN-15A)

2 g of enoxaparin (solid) were dissolved in 100 ml of a 0.1 molar HEPES buffer pH 8.8. While stirring at 24° C., a prepared suspension prepared from 2.38 g of bis (2-aminoethyl)amine-N,N,N′,N″,N″-pentaacetic acid dianhydride (DTPA anhydride) and 6.8 ml of dimethyl sulfoxide, corresponding to a 15-fold molar excess of reagent over the amount of enoxaparin introduced was added dropwise.

During the addition, the pH was monitored and kept at pH 8.8 by metering in 1 molar sodium hydroxide solution. The reaction mixture was vigorously stirred at room temperature for 30 minutes, keeping the pH constant during this, if necessary, by further addition of 1 molar sodium hydroxide solution. 2.48 g of solid gadolinium(III) chloride hexahydrate, likewise corresponding to a 15-fold molar excess over the enoxaparin present, were then stirred into the mixture. The pH was adjusted to 6.5 by titration with 1 molar hydrochloric acid. The reaction mixture was stirred further at 4° C. for 24 hours. The modified enoxaparin fraction was desalted and freed of unreacted reagent by dialysis for 24 hours against a total of 4 volumes each of 5 l of water in a commercially available dialysis tube (molecular weight separation limit 1 000). During this, the initially introduced water was replaced by fresh water after 1 hour, 3 hours and 16 hours. The contents of the tube were then freeze-dried. 1.77 g of a modified enoxaparin were obtained.

The product was preferably purified once again by gel filtration on Sephadex® G-25 using pyrogen-free water. The factor Xa inhibition test showed an inhibitory strength comparable with that of the original enoxaparin. Analysis of the gadolinium incorporation by inductively coupled plasma atomic emissions spectrometry revealed a content of 5 mol of gadolinium per mol of enoxaparin.

### EXAMPLE 3a

### Preparation of an Enoxaparin Derivative (EN-15B)

2 g of enoxaparin (solid) were dissolved in 100 ml of a 0.1 molar HEPES buffer pH 8.8. While stirring at 24° C., a prepared suspension prepared from 2.38 g of bis(2-aminoethyl)amine-N,N,N′,N″,N″-pentaacetic acid dianhydride (DTPA anhydride) and 6.8 ml of dimethyl sulfoxide, corresponding to a 15-fold molar excess of reagent over the amount of enoxaparin introduced was added dropwise. During the addition, the pH was monitored and kept at pH 8.8 by metering in 1 molar sodium hydroxide solution. The reaction mixture was vigorously stirred at room temperature for 30 minutes, keeping the pH constant during this, if necessary, by further addition of 1 molar sodium hydroxide solution. 2.48 g of solid gadolinium(III) chloride hexahydrate, likewise corresponding to a 15-fold molar excess over the enoxaparin present, were then stirred into the mixture. The pH was adjusted to 6.5 by titration with 1 N hydrochloric acid. The reaction mixture was stirred further at 4° C. for 24 hours. After this period, 0.4 ml of ethanolamine was added and the mixture was stirred at room temperature for a further 30 minutes. Finally, the pH was adjusted to 7.0 by adding hydrochloric acid, and the reaction mixture was precipitated by diluting with 4 times the volume of methanol. The precipitate was filtered off on a suction filter and redissolved at high concentration in 30 ml of pure water.

The modified enoxaparin was desalted and freed of unreacted reagent by dialysis for 24 hours against a total of 4 volumes each of 0.5 l of water in a commercially available dialysis tube (molecular weight separation limit 1 000). During this, the initially introduced water was replaced by fresh water after 1 hour, 3 hours and 16 hours. The contents of the tube were then freeze-dried.

It is possible to save time for the desalting of relatively small volumes by using a commercially available gel filtration column (e.g., Pharmacia HiPrep® desalting). The desalted fraction is then freeze-dried in the same way.

1.86 g of a modified enoxaparin were obtained. The factor Xa inhibition test showed that the inhibitory strength was undiminished compared with the original enoxaparin. Analysis of the gadolinium incorporation by inductively coupled plasma atomic emissions spectrometry revealed a content of 2.7 mol of gadolinium per mol of enoxaparin.

### EXAMPLE 4

Enoxaparin derivatives with a 3-fold, 8-fold and 50-fold excess of DTPA anhydride and gadolinium(III) chloride hexahydrate were also prepared as in example 2. The compounds are referred to hereinafter as EN 3, EN8 and EN-50 for short. A product which was reacted with a 50-fold excess of DTPA anhydride but not subsequently loaded with gadolinium ions was prepared likewise as in example 2. This product, referred to as EN-50Z hereinafter, corresponds to an enoxaparin derivative with increased anionic charge and correspondingly modulated pharmacological properties, but which is also of interest in particular because it can also be used in a simple manner as chelating precursor for loading with other, e.g. also reactive, cations.

### EXAMPLE 5

### Preparation of a Heparin Derivative (He-15B)

2 g of commercially available heparin sodium (solid; Sigma H4784) were dissolved in 100 ml of a 0.1 molar HEPES buffer pH 8.8. While stirring at 24° C., a prepared suspension prepared from 1.19 g of bis(2-aminoethyl)amine-N,N,N′,N″,N″-pentaacetic acid dianhydride (DTPA anhydride) and 3.4 ml of dimethyl sulfoxide, was added dropwise. During the addition, the pH was monitored and kept at pH 8.8 by metering in 1 molar sodium hydroxide solution. The reaction mixture was vigorously stirred at room temperature for 30 minutes, keeping the pH constant during this, if necessary, by further addition of 1 molar sodium hydroxide solution. 1.24 g of solid gadolinium(III) chloride hexahydrate were then stirred into the mixture. The pH was adjusted to 6.5 by titration with 1 N hydrochloric acid. After this period, 0.2 ml of ethanolamine was added and the mixture was stirred at room temperature for a further 30 minutes. Finally, the pH was adjusted to 7.0 by adding hydrochloric acid, and the reaction mixture was precipitated by diluting with 4 times the volume of methanol. The precipitate was filtered off on a suction filter and redissolved at high concentration in 30 ml of pure water.

The modified heparin was desalted and freed of unreacted reagent by dialysis for 24 hours against a total of 4 volumes each of 0.5 l of water in a commercially available dialysis tube (molecular weight separation limit 1,000). During this, the initially introduced water was replaced by fresh water after 1 hour, 3 hours and 16 hours. The contents of the tube were then freeze-dried.

It is possible to save time for the desalting of relatively small volumes by using a commercially available gel filtration column (e.g., Pharmacia HiPrep® desalting). The desalted fraction is then freeze-dried in the same way.

1.65 g of a modified heparin were obtained. The factor Xa inhibition test showed that the inhibitory strength was undiminished compared with the original heparin. Analysis of the gadolinium incorporation by inductively coupled plasma atomic emissions spectrometry revealed a content of 1.9 mol of gadolinium per mol of heparin.

### EXAMPLE 6

### Test System for Investigating the Heparinoid-Dependent Inhibition of Factor Xa Principle of the Test

On addition of antithrombin III and an excess of factor Xa to a test sample containing a heparinoid, the heparinoid in the test sample, which is bound with antithrombin III to give a complex, inactivates factor Xa. The remaining activity of factor Xa can be measured using a synthetic chromogenic substrate. In this case, para-nitroaniline is liberated from the substrate by enzymatic cleavage and can be detected by photometry through measurement of the change in extinction at a wavelength of 405 nm per unit time. The amount of liberated para-nitroaniline is inversely proportional to the concentration of the heparinoid in the test sample (Teien M. L. et al., Thromb. Res. 8 (3), 413-6 [1976]). A calibration series is constructed with graduated concentrations of the heparinoid in the medium investigated (change in extinction per unit time as a function of the concentration). The concentration of the heparinoid can be found by comparison from the change in extinction of a test sample per unit time.

### Test Procedure:

The calibration lines are preferably constructed using a heparinoid concentration range from 0.5 μg/ml to 3 μg/ml. The samples in this concentration series are diluted 1:10 with 0.046 M Tris buffer pH 8.4, which contains 0.15 M NaCl, 0.007 M EDTA, 0.1% Tween 80 and 0.12 IU of human antithrombin III. 50 μl portions of the diluted samples are incubated with 50 μl of bovine factor Xa (13.6 U/ml) at 37° C. for 80 seconds. Then 50 μl of 1.1 mM chromogenic substrate S-2765 are added. The change in extinction at a wavelength of 405 nm per minute is measured in a photometer. 50 μl portions of the suitably prediluted test samples are treated according to the same pattern.

### Table 1 Shows the Results:

TABLE 1

| Concentration | | | | | | |
|---|---|---|---|---|---|---|
| (μg/ml)* | Enoxaparin | EN-3 | EN-8 | EN-15 | EN-50 | EN-50Z |
| 0.3 | 0.473 | 0.431 | 0.463 | 0.406 | 0.362 | 0.439 |
| 0.25 | 0.545 | 0.491 | 0.509 | 0.486 | 0.455 | 0.484 |
| 0.2 | 0.607 | 0.544 | 0.579 | 0.577 | 0.519 | 0.558 |
| 0.15 | 0.671 | 0.659 | 0.656 | 0.63 | 0.602 | 0.643 |
| 0.1 | 0.728 | 0.711 | 0.737 | 0.71 | 0.692 | 0.726 |
| 0.05 | 0.806 | 0.794 | 0.808 | 0.786 | 0.793 | 0.794 |
| 0 | 0.884 | 0.897 | 0.897 | 0.896 | 0.907 | 0.910 |

### EXAMPLE 7

### Aggrecanase Test System

The test is carried out in the 96-well microtiter plate format.

A dilution series of the labeled enoxaparin is made up in pure water for preparation.

### Digestions:

A predetermined amount of synovial fluid or aggrecanase activity, which brings about an extinction of from 1.0 to 1.4 at 405 nm under the test conditions, is mixed in each well with 3 μl of the respective dilution of labeled enoxaparin, made up to a final volume of 300 μl with Dulbecco's modified Eagle medium (DMEM) and incubated in a CO₂ cell culture incubator at 37° C. for 1 hour. Then 5 μl of a solution of 1 μg/μl Agg1mut substrate (as disclosed in: Bartnik E. et al., EP 785274 (1997); substrate in DMEM) are added to each well, and the mixture is digested in a CO₂ incubator at 37° C. for 4 hours.

### Preparation of the Test Plate:

In the first step, each well is coated with 100 μl of a solution of commercially available anti-mouse immunoglobulin G (from goat; 5 μg/ml in physiological phosphate buffer pH 7.4 [PBS buffer]) at room temperature for 1 hour. After the plate has been washed with PBS buffer with the addition of 0.1% Tween 20 (called washing buffer hereinafter), each well is blocked with 100 μl of a solution of 5% bovine serum albumin in PBS buffer with the addition of 0.05% Tween 20 at room temperature for 1 hour.

After renewed washing with washing buffer, each well is incubated with 100 μl of a 1:1 000-diluted solution of BC-3 antibody in PBS buffer with 0.05% Tween 20 and 0.5% bovine serum albumin at room temperature for 1 hour; this antibody recognizes aggrecanase-typical cleavage fragments (Hughes C. E. et al., Biochem. J. (1995), 305 (3), 799-804).

### Test Procedure:

After the test plate has been washed with washing buffer, the complete mixture from the preceding digestion is transferred well for well to the test plate and incubated at room temperature for 1 hour. After the plate has been washed with washing buffer, 100 μl of the second antibody (goat anti-human IgG, peroxidase-labeled, 1:1 000 in 0.5% BSA/PBS buffer/0.05% Tween 20) are added, and incubation with this is again carried out at room temperature for 1 hour. After renewed washing with washing buffer, color development takes place by addition of 100 μl of ABTS substrate solution (2 mg/ml 2,2′-azinobis(3-ethylbenzothiazolinesulfonic acid in 40 mM sodium citrate with 60 mM disodium hydrogen phosphate, adjusted to pH 4.4 with acetic acid; 0.25 ml of 35% hydrogen peroxide added per ml immediately before the measurement). The measurement takes place in the shaking mode at 405 nm against a reference filter (620 nm) with automatic readings at 5-second intervals. The test is stopped as soon as a maximum extinction (405 nm) in the range from 1.0 to 1.4 is reached.

TABLE 2
(Substrate conversion in % compared with the conversion of noninhibited
aggrecanase)

| Concentration | | | | EN- | EN- | EN- | HE- |
|---|---|---|---|---|---|---|---|
| (μg/ml) | Enoxaparin | EN-3 | EN-8 | 15 | 50 | 50Z | 15B |
| 100 | 52.1 | 31.2 | 26.6 | 32.2 | 38.3 | 8.9 | 23.4 |
| 10 | 42.2 | 20.2 | 20.8 | 28.5 | 47.0 | 15.4 | 16.8 |
| 1 | 53.8 | 36.9 | 43.7 | 37.6 | 29.4 | 36.4 | 12.4 |
| 0.1 | 99.1 | 87.6 | 97.5 | 89.5 | 88.4 | 72.9 | 67.7 |
| 0.01 | 116.4 | 113.2 | 109.8 | 106.0 | 108.6 | 89.9 | 92.1 |
| 0.001 | 118.0 | 115.3 | 110.0 | 109.5 | 111.2 | 95.8 | 95.0 |
| 0.0001 | 107.8 | 107.2 | 106.7 | 101.0 | 102.3 | 98.2 | 99.3 |

### EXAMPLE 8

### Magnetic Resonance Imaging Experiment

The heparinoid derivatives EN-3, EN-8, EN-15 and EN-50 of the invention which are described in examples 3 and 4 are dissolved in distilled water in concentrations of 0.01, 0.1, 1.0 and 10.0 mM and introduced into Eppendorf tubes with a capacity of 0.5 milliliter. Each tube is inserted into a larger Eppendorf tube which has a capacity of 1.5 milliliters and is filled with distilled water. The latter tubes are arranged in a plastic rack and imaged in a magnet resonance imaging system from Bruker Medical GmbH, Ettlingen, at a magnetic field strength of 7 tesla. To describe the effect of the heparinoid derivatives of the invention on the relaxation times of the water protons in the solutions, MR images differing in contrast characteristics are measured using the Paravision® software developed by Bruker Medical GmbH. In order to see all the tubes simultaneously in the image, a coronal (=horizontal) slice plane with a layer thickness of 2 mm is chosen.

In T1-weighted spin-echo images (echo time TE=13 msec, relaxation time TR=100 msec, 1 echo, NA=2, matrix 2562), the tubes show signal loss with all the derivatives at a concentration of 10 mM, and with EN-50 and EN-15 there is even distortion of the image because of the local impairment of the homogeneity of the magnetic field due to the higher gadolinium concentration per mol of enoxaparin. There is a signal enhancement by a factor of about 10 in relation to the signal of the surrounding distilled water in the tubes with EN-50 and EN-15 at a concentration of 0.1 mM and those with the derivatives EN-8 and EN-3 at a concentration of 1.0 mM. The signal enhancement in the remaining tubes decreases in accordance with the lower concentration of the heparinoid derivatives of the invention and the lower relative gadolinium concentration.

The T2 relaxation time for the individual solutions is determined in a spin-echo experiment with 16 individual echoes (TE 13 msec, TR=3 000 msec, 16 echoes, NA=1, matrix 2562). The results are summarized numerically in table 3. In all 10 mM solutions of the heparinoid derivatives of the invention the T2 time is shortened so much that it can no longer be reliably determined. Based on the T2 time for pure distilled water (907 msec) determined under these measurement conditions, a maximum shortening by a factor of 60 is reached. The shortening of the T2 time is proportional to the concentration of the heparinoid derivatives of the invention and the individual relative gadolinium concentration.

### Summary:

The MRI investigation shows that the heparinoid derivatives of the invention shorten, depending on their concentration and depending on the molar ratio of gadolinium to enoxaparin, the T1, T2 and T2* relaxation times of the water protons of solutions containing the heparinoid derivatives of the invention, so that in the T1-weighted MRI images there is an increase in the signal intensity or even a partial or total signal loss.

TABLE 3
(T2 - Relaxation times)

| Derivative | 0.01 mM | 0.1 mM | 1.0 mM | 10 mM | Water_{dist} |
|---|---|---|---|---|---|
| EN-50 | 201 | 23 | nd* | nd* | 995 |
| EN-15 | 347 | 47 | nd* | nd* | 871 |
| EN-8 | 648 | 124 | 14.4 | nd* | 845 |
| EN-3 | 682 | 124 | 14.3 | nd* | 917 |

(*nd = T2 cannot be determined reliably because too short)

Measured T2 times (in msec) of the water protons in solutions of the heparinoid derivatives of the invention in the stated millimolar concentrations. The T2 times are determined using a spin-echo imaging sequence with 32 individual echoes. The average of the T2 relaxation in the surrounding distilled water is 907 msec.

### EXAMPLE 9

### Magnetic Resonance Imaging Experiment

The isolated knee joint of a pig (weight about 40 kg, 4 months old) is exposed. The joint is fastened in a plastic container in such a way that only one condyle is immersed in a solution of EN-15A (0.1 mM in physiological saline, room temperature). A T1-weighted spin-echo image with high spatial resolution (voxel size about 140×180 μm, layer thickness 2 mm) is recorded every 30 minutes over a period of 14 hours. The image slice is approximately sagittal through the condyle and bone shaft and shows the region of the trabecular bone gray to black surrounded by a pale gray layer of cartilage, which reaches a thickness of up to 7 mm. The bone/bone and cartilage/surrounding solution interfaces are clearly evident. Some hours after immersion in the EN-15A solution there is a change in the appearance of the outer layer of cartilage. New laminar structures form and run approximately parallel to the surface of cartilage: a first thin (about 140 μm deep) hyperintense layer near the surface, followed by a broader (about 500 μm deep) hypointense layer. Signal loss takes place increasingly in this second layer and can be explained both by an increased concentration of EN-15A and by a greatly reduced mobility of the EN-15A in this cartilage zone, because both effects are able to induce such a rapid fall in the signal that a signal is no longer measurable. This is followed by a third hyperintense layer which has a similar width and brightness to the first. A fourth, highly hyperintense layer which is about twice to three times as broad as the second follows. Yet a fifth layer is to be seen subsequently, its signal intensity corresponding approximately to that of the first or third layer, although being somewhat broader than the latter. The distance from the cartilage surface of the deepest front of EN-15A is about 1.6 to 1.7 mm.

### SUMMARY

The MRI investigation shows that the heparinoid derivatives of the invention penetrate into the native intact cartilage of an isolated femoral condyle of a pig, with the heparinoid derivatives of the invention bringing about a change in the MR signal intensity because of the effect on the T1, T2 and T2* relaxation times of the water protons in the cartilage; this change in the T1, T2 and T2* relaxation times of the water protons is enhanced by the reduced mobility of the heparinoid derivatives of the invention inside the cartilage.

We claim:

1. A process for preparing a heparinoid derivative, said heparinoid derivative comprising
a heparinoid selected from the group consisting of enoxaparin, nadroparin, dalteparin, bemiparin, tinzaparin and ardeparin;
a chelating agent which is covalently bonded to the heparinoid, said chelating agent being selected from the group consisting of diethylenetriamine-N,N,N′,N″,N″-pentaacetic acid dianhydride, 1,2-bis(2-aminoethoxyethane)-N,N,N′,N′-tetraacetic acid, ethylenediamine-N,N,N′,N′-tetraacetic acid, 1,4,7,10-tetraazacyclododecane-1,4,7,10-tetraacetic acid, 1,4,7,10-tetraazacyclododecane-1,4,7-triacetic acid, nitrilotriacetic acid, triethylenetetraminehexaacetic acid, 4-carboxy-5,8,11-tris(carboxymethyl)-1-phenyl-2-oxa-5,8,11-triazamidecan-13-oic acid and N,N′-bis(pyridoxal 5-phosphate)-ethylenediamine-N,N′-diacetic acid; and
a paramagnetic metal cation selected from the group consisting of Sc, Ti, V, Cr, Mn, Fe, Co, Ni, Cu, Mo, Ru, La, Ce, Pr, Nd, Pm, Sm, Eu, Gd, Tb, Dy, Ho, Er, Tm and Yb,
said process comprising reacting a heparinoid with an activated chelating agent to produce a covalently bonded heparinoid chelate, and without further purification of the heparinoid chelate, adding a paramagnetic metal cation to the covalently bonded heparinoid chelate, wherein the activated chelating agent is employed in an excess of from 1:1 to 50:1 in relation to the initial heparinoid, based on the molecular weight.

2. The process of claim 1, wherein the activated chelating agent is employed in an excess of from 1.5:1 to 15:1, based on the molecular weight.

3. The process of claim 1, wherein the activated chelating agent is an anhydride.

4. The process of claim 3, wherein the activated chelating agent is diethylenetriamine-N,N,N′,N″,N″-pentaacetic acid dianhydride.

5. The process of claim 1, wherein the paramagnetic metal cation employed is Gd³⁺ in a salt form of gadolinium(III) chloride hexahydrate or gadolinium(III) acetate hydrate.

6. The process of claim 1, wherein the ratio of heparinoid chelate to paramagnetic metal cation on addition is from 1:1 to 1:50 based on the molecular weight.

7. The process of claim 6, wherein the ratio of heparinoid chelate to paramagnetic metal cation on addition is from 1:1.5 to 1:15, based on the molecular weight.

8. The process of claim 1, wherein the heparinoid derivative which is obtained is purified and desalted for further use by dialysis or gel filtration or both, and optionally is subjected to freeze-drying.
